(19) United States
(12) Patent Application Publication
(10) Pub. No.: US 2003/0111077 A1
(43) Pub. Date: Jun. 19, 2003

(54) Patient humidification systems

(76) Inventors: Theron Van Hooser, Salt Lake City, UT (US); L. John Teuscher, Springville, UT (US)

Correspondence Address: KIMBERLY-CLARK WORLDWIDE, INC., 401 NORTH LAKE STREET, NEENAH, WI, 54956

(21) Appl. No.: 10/023,087

(22) Filed: Dec. 17, 2001

(51) Int. Cl. A61M 15/00

(52) U.S. Cl. 128/203.16; 128/203.12

(57) ABSTRACT

A system to provide inspiratory air to a patient having a humidification mechanism including an inspiratory gas inlet and an inspiratory gas outlet in fluid communication with the inspiratory gas inlet. The humidification mechanism may be adapted to humidify inspiratory air flowing into the inspiratory gas inlet and exiting the humidification mechanism through the inspiratory gas outlet. A patient inspiratory gas tube is provided to extend between the inspiratory gas outlet of the humidification mechanism and a patient, the patient inspiratory gas tube being adapted to provide inspiratory gas to a patient. A patient expiratory gas tube may provide a flow path for expiratory gas from a patient. A shroud may be disposed about at least a portion of the humidification mechanism, the shroud including an expiratory gas inlet and an expiratory gas outlet, the patient expiratory gas tube being attached to the expiratory gas inlet of the shroud so that expiratory gas from a patient flows into the shroud through the expiratory gas inlet and exits the shroud through the expiratory gas outlet.

The present invention relates generally to humidification of gases within ventilating systems that provide mechanical ventilation to a patient.

Humidifiers are an important part of ventilator breathing circuits because breathing gas must be warm and humidified for optimal inhalation therapy. Humidification is particularly important for patients treated with ventilators for prolonged periods. Available respiratory humidifiers can generally be placed in one of three groups according to their method of operation: nebulizing, bubbling and heated evaporation. Nebulizing humidifiers rely on the flow of pressurized breathing gas through an ejector-like element to generate an aerosol spray. With bubbling humidifiers, breathing gas is forced directly through a pool of liquid water. Heated evaporation humidifiers employ a heated contact chamber, where breathing gas is passed over heated water to absorb water vapor.

A persistent, serious problem with humidifiers is condensation or “rain-out” in the ventilator circuit downstream of the humidifier. As humidified breathing gas travels through the tubing of the ventilation system, it is cooled by ambient air. If the breathing gas is over-humidified or if the room temperature drops, condensation occurs. Such condensation is considered contaminated waste and presents a disposal problem. Therefore, tubing cleanup and system readjustments become all too frequent steps in the maintenance of ventilator circuits. System configurations that reduce the opportunity for condensation in the ventilator system enhance the usability of the system both in the hospital and home care environments.

Various embodiments of the present invention relate to a system to provide inspiratory air to a patient having a humidification mechanism that may include an inspiratory gas inlet and an inspiratory gas outlet. The inspiratory gas outlet of the humidification mechanism is in fluid communication with the inspiratory gas inlet of the humidification mechanism. The humidification mechanism is adapted to humidify inspiratory air flowing into the inspiratory gas inlet and exiting the humidification mechanism through the inspiratory gas outlet. In selected embodiments, a controller may be provided that is adapted to control the humidification of the inspiratory air exiting the humidification mechanism.

A patient inspiratory gas tube may be provided that is adapted to extend between the inspiratory gas outlet of the humidification mechanism and a patient to provide a flow path for inspiratory gas to a patient. A patient expiratory gas tube may also be provided that is adapted to provide a flow path for expiratory gas from a patient. In particular embodiments, the patient inspiratory gas tube may be disposed at least partially within the patient expiratory gas tube. The patient inspiratory gas tube may also be disposed completely within the patient expiratory gas tube.

A shroud may be disposed about at least a portion of the humidification mechanism, the shroud including an expiratory gas inlet and an expiratory gas outlet. The patient expiratory gas tube may be attached to the expiratory gas inlet of the shroud so that expiratory gas from a patient flows into the shroud through the expiratory gas inlet and exits the shroud through the expiratory gas outlet. In some embodiments, the shroud may be formed from a flexible and/or transparent plastic such as, for example, polyurethane or ethylvinylacetate (EVA).

In such a system, a controller may be utilized to control the temperature of the inspiratory air exiting the humidification mechanism.

A ventilator inspiratory tube may be provided that is adapted to extend between the inspiratory gas inlet of the humidification mechanism and a ventilator. Similarly, a ventilator expiratory tube may also be provided that is adapted to extend between the expiratory gas outlet of the shroud and a ventilator.

Selected embodiments of the present invention include a shroud for use in a system which provides mechanical ventilation to a patient, the shroud including an expiratory gas inlet adapted to receive expired air from a patient and an expiratory gas outlet adapted to permit the expired air within the shroud to pass exteriorly of the shroud. In such an embodiment, the shroud may be adapted to be disposed about a humidifier that is configured to humidify inspiratory air flowing to a patient.

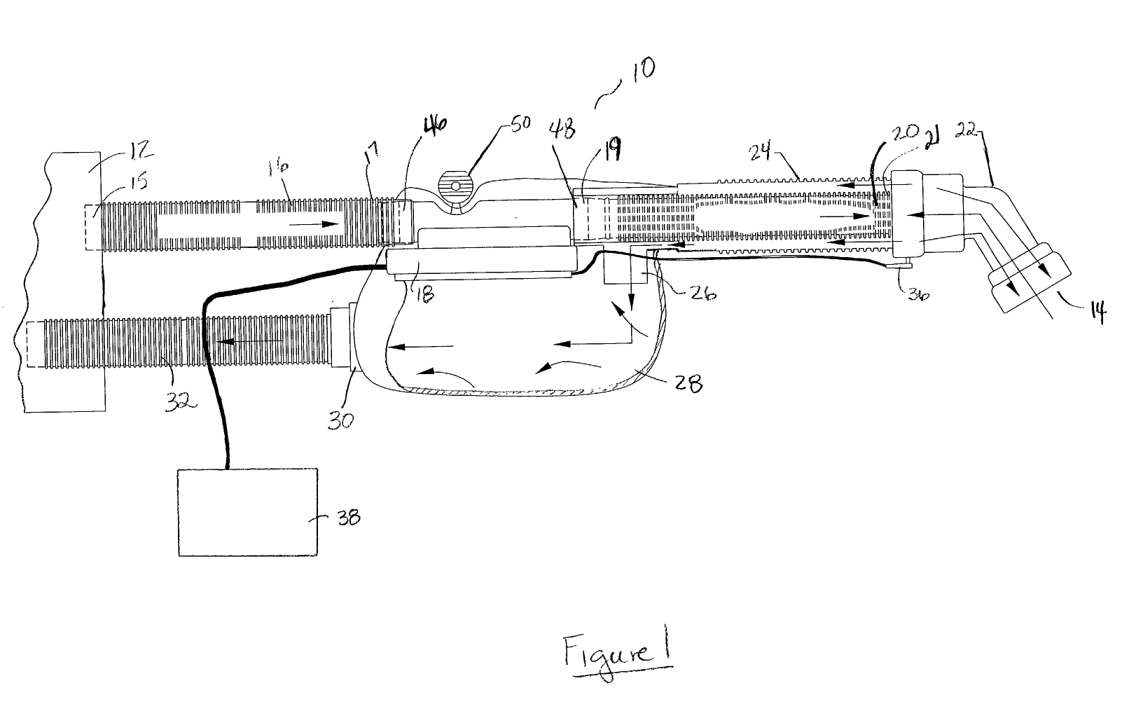

FIG. 1 is a schematic representation of the present invention.

A system that may be used in association with a mechanical ventilator for a patient is shown in FIG. 1 at 10. The system 10 shown therein includes a ventilator 12 that provides a source of breathing gas to a patient positioned at the end 14 of the system 10. As indicated by the arrows in FIG. 1, breathing gas flows from the ventilator 12 through a ventilator inspiratory gas tube 16 and into a humidification mechanism 18. The breathing gas exits the humidification mechanism 18 through a patient inspiratory gas tube 20 and into a patient connector 22. Expiratory and inspiratory gases flow to and from the patient through the patient connector 22. Expiratory gases flow from the patient through the patient connector 22 and through the patient expiratory gas tube 24. The expiratory gases then flow through an expiratory gas inlet 26 into a shroud 28 that is disposed about at least a portion of the humidification mechanism 18. Expiratory gases flow out of the shroud 28 through an expiratory gas outlet 30 and into a ventilator expiratory gas tube 32. A controller 38 may be included as part of the system 10 to control the temperature of the water within the humidification mechanism 18 and the inspiratory gases.

The humidification mechanism 18 may be variously configured and may provide both heat and humidity to the breathing gas. A controller 38 may be connected to the humidification mechanism 18. Various types of humidification mechanisms may be used in the present invention, including the humidifier disclosed in U.S. Pat. No. 6,095,505 that issued on Aug. 1, 2000, which is hereby incorporated by reference in its entirety. As disclosed therein, the humidifier is a heated evaporation humidifier that includes an evaporation module, a flow controller, and a temperature controller. The evaporation module may include a contact chamber defined by a rigid housing and in part by a flash resistant heat exchanger. A wicking layer is positioned in contact with the heat exchanger, and serves to receive and distribute liquid water entering the contact chamber. An electric resistive heater is provided for the heat exchanger so as to evaporate liquid water arriving in the contact chamber from a liquid water source. So that breathing gas may pass through the evaporation module, the rigid housing includes a gas inlet and a breathing gas outlet. A rigid housing also includes a water inlet for receiving water from water source through a liquid water flow passageway.

Referring again to FIG. 1, the ventilator 12 that provides a source of breathing gas to a patient is shown at the left of the figure. The ventilator inspiratory gas tube 16 has two ends 15 and 17, the end 15 of the ventilator inspiratory gas tube 16 being attached to the ventilator 12 so that breathing gas may exit the ventilator 12 and flow through the ventilator inspiratory gas tube 16. The end 17 of the ventilator inspiratory gas tube 16 is attached to the inspiratory gas inlet 46 of the humidification mechanism 18 so that the breathing gas may flow through the ventilator inspiratory gas tube 16 and into the inspiratory gas inlet 46 of the humidification mechanism 18. The tubing ends, connectors and the shroud may be variously secured to each other by a variety of mechanisms, including, for example, snap-fit using retention barbs and the like, force-fit, adhesive, and the like.

The breathing gas that flows into the inspiratory gas inlet 46 of the humidification mechanism 18 is humidified to a predetermined level before leaving the humidification mechanism 18 through the inspiratory gas outlet 48. The controller 38 may be adapted to permit an operator to set and achieve desired parameters for the heat and humidification of the inspiratory gas that flows from the humidification mechanism 18. Various configurations of the controller 38 may be used to control the heat and humidification of the inspiratory air being provided to the patient. In one such configuration and as shown in FIG. 1, a temperature probe 36 may be disposed proximate to a patient connector 22, the temperature probe being configured to feed information regarding temperature levels to the humidification mechanism 18 and/or the controller 38. As shown in FIG. 1, a single block may be used to indicate several individual components that may collectively perform the various functions that are required.

The humidification mechanism 18 may include a support member 50, which is depicted in FIG. 1 as a sphere having a segment of the sphere removed, although a variety of other configurations of support members may be used with the present invention. The support member 50 may be attached to another structure to provide support to the humidification mechanism 18.

The breathing gas exits the humidification mechanism 18 through a patient inspiratory gas tube 20, which includes two ends, 19 and 21. The end 19 of the patient inspiratory gas tube 20 is attached to the inspiratory gas outlet 48 of the humidification mechanism 18. The end 21 of the patient inspiratory gas tube 20 is connected to the patient connector 22.

The patient connector 22 permits the patient to inhale inspiratory gas that is provided through the patient inspiratory gas tube 20, and exhale expiratory gas into the patient expiratory gas tube 24, both of which are connected to the patient connector 22. In some embodiments, a “Y” connector may be utilized. In other embodiments, a straight or 90 degree connector may be utilized.

The patient connector 22 may be attached to any of a variety of mechanisms that enable a patient to breathe the inspiratory air being provided by the ventilator. For example, artificial airways such as tracheostomy tubes and endotracheal tubes may be connected to the patient connector 22. Alternately, a variety of masks that are adapted to fit over a patient's nose and mouth may be attached to the patient connector 22.

As seen in FIG. 1, the patient inspiratory gas tube 20 may be disposed within the patient expiratory gas tube 24. Such a coaxial arrangement of tubes reduces the opportunity for rain-out to occur, as the expired gases within the patient expiratory gas tube 24 act to insulate the patient inspiratory gas tube 10 and maintain the temperature of the inspiratory gases flowing through the patient inspiratory gas tube 10.

A shroud 28 is provided which surrounds at least a portion of the humidification mechanism 18. As seen in FIG. 1, the shroud 18 may form a part of the expiratory air flow pathway in that the expiratory gas may directly contact the interior of the shroud 18. In such an embodiment, the shroud 18 may be disposable. Alternately, the shroud 18 may include a disposable liner.

As shown in FIG. 1, the expiratory gas exits the patient expiratory gas tube 24 and enters the shroud 18 through the expiratory gas inlet 26. As the expiratory gas exhaled by a patient may be warmer than the ambient air of the patient's environment, the expired gases may help to insulate the humidification mechanism 18 from the cooler air. In this manner, the likelihood or amount of condensation or “rain-out” within the tubing may be reduced.

The expiratory gas flows out of the shroud 18 through the expiratory gas outlet 30 and into the ventilator expiratory gas tube 32.

The invention may be embodied in other specific forms without departing from the scope and spirit of the inventive characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

We claim:

1. A system to provide inspiratory air to a patient comprising:
a humidification mechanism comprising
an inspiratory gas inlet, and
an inspiratory gas outlet in fluid communication with the inspiratory gas inlet, the humidification mechanism adapted to humidify inspiratory air flowing into the inspiratory gas inlet and exiting the humidification mechanism through the inspiratory gas outlet;
a patient inspiratory gas tube that is adapted to extend between the inspiratory gas outlet of the humidification mechanism and a patient, the patient inspiratory gas tube adapted to provide inspiratory gas to a patient;
a patient expiratory gas tube adapted to provide a flow path for expiratory gas from a patient;
a shroud disposed about at least a portion of the humidification mechanism, the shroud comprising an expiratory gas inlet and an expiratory gas outlet, the patient expiratory gas tube being attached to the expiratory gas inlet of the shroud so that expiratory gas from a patient flows into the shroud through the expiratory gas inlet and exits the shroud through the expiratory gas outlet.

2. The humidification system of claim 1 further comprising a controller adapted to control the humidification of the inspiratory air exiting the humidification mechanism.

3. The humidification system of claim 1 wherein the patient inspiratory gas tube is disposed at least partially within the patient expiratory gas tube.

4. The humidification system of claim 1, further comprising a ventilator inspiratory tube that is adapted to extend between the inspiratory gas inlet of the humidification mechanism and a ventilator.

5. The humidification system of claim 1, further comprising a ventilator expiratory tube that is adapted to extend between the expiratory gas outlet of the shroud and a ventilator.

6. The humidification system of claim 1, the shroud being formed of a flexible plastic.

7. The humidification system of claim 1, the shroud being formed of a transparent plastic.

8. A humidification system comprising:
a humidification mechanism comprising
an inspiratory gas inlet,
an inspiratory gas outlet in fluid communication with the inspiratory gas inlet, the humidification mechanism adapted to humidify inspiratory air flowing into the inspiratory gas inlet and exiting the humidification mechanism through the inspiratory gas outlet;
a patient inspiratory gas tube that is adapted to extend between the inspiratory gas outlet of the humidification mechanism and a patient, the patient inspiratory gas tube adapted to provide inspiratory gas to a patient;
a shroud disposed about at least a portion of the humidification mechanism, the shroud comprising an expiratory gas inlet and an expiratory gas outlet;
a patient expiratory gas tube that is adapted to extend between a patient and the expiratory gas inlet of the shroud, the patient inspiratory gas tube being disposed at least partially within the patient expiratory gas tube, the expiratory gas tube being adapted to provide a flow path for expiratory gas from a patient to the shroud; and
whereby expiratory gas flows from the shroud through the expiratory gas outlet.

9. The humidification system of claim 8, further comprising a controller adapted to control the humidification of the inspiratory air exiting the humidification mechanism.

10. The humidification system of claim 8, further comprising a ventilator inspiratory tube that is adapted to extend between the inspiratory gas inlet of the humidification mechanism and a ventilator.

11. The humidification system of claim 8, further comprising a ventilator expiratory tube that is adapted to extend between the expiratory gas outlet of the shroud and a ventilator.

12. A shroud for use in a system which provides mechanical ventilation to a patient, the shroud comprising:
an expiratory gas inlet adapted to receive expired air from a patient;
an expiratory gas outlet adapted to permit the expired air within the shroud to pass exteriorly of the shroud;
whereby the shroud is adapted to be disposed about a humidifier adapted to humidify inspiratory air flowing to a patient.

13. The shroud of claim 12 being formed of a flexible material.

14. The shroud of claim 12 being formed of a transparent material.
